(19) United States
(12) Patent Application Publication
(10) Pub. No.: US 2018/0078414 A1
(43) Pub. Date: Mar. 22, 2018

(54) INCISION APPARATUS WITH STABILIZING CAP

(71) Applicant: V.R. HEXIS LTD., Tel Aviv (IL)

(72) Inventors: Josef DELI, Karmiel (IL); Avraham RINOT, Tel Mond (IL)

(73) Assignee: V.R. HEXIS LTD., Tel Aviv (IL)

(21) Appl. No.: 15/563,592

(22) PCT Filed: Mar. 31, 2016
(86) PCT No.: PCT/IL2016/050353

§ 371 Date: Sep. 30, 2017

Related U.S. Application Data
(60) Provisional application No. 62/141,878, filed on Apr. 2, 2015.

(51) Int. Cl. A61F 9/007

(52) U.S. Cl. CPC A61F 9/00754; A61B 2217/005

(57) ABSTRACT

An apparatus for making an incision in a surface has a cutting head that includes a stabilizing cap for placement on the surface and operable to inhibit relative motion between the cutting head and the surface. A cutting element is located radially distal to the stabilizing cap and is configured to make the incision which is radially distal to and at least partially surrounds the stabilizing cap.

### FIELD OF THE INVENTION

The present invention relates to an incision apparatus. More particularly, the present invention relates to an incision apparatus with a stabilizing cap.

### BACKGROUND OF THE INVENTION

It is sometimes necessary to manipulate a tool or instrument to make a small cut or incision within a confined space. For example, in some types of eye surgery, such as cataract surgery, an instrument is needed for cutting through the capsule of the lens. The instrument is inserted to the lens through a small slit or cut that is made in the cornea.

### SUMMARY OF THE INVENTION

There is thus provided, in accordance with an embodiment of the present invention, an apparatus for making an incision in a surface, the apparatus including a cutting head that includes: a stabilizing cap for placement on the surface and operable to inhibit relative motion between the cutting head and the surface; and a cutting element that is located radially distal to the stabilizing cap and that is configured to make the incision which is radially distal to and at least partially surrounds the stabilizing cap.

Furthermore, in accordance with an embodiment of the present invention, a surface of the stabilizing cap for placement on the surface is concave.

Furthermore, in accordance with an embodiment of the present invention, the stabilizing cap is substantially circular.

Furthermore, in accordance with an embodiment of the present invention, the stabilizing cap has a noncircular shape.

Furthermore, in accordance with an embodiment of the present invention, the stabilizing cap includes at least one port that is connectable to a source of suction.

Furthermore, in accordance with an embodiment of the present invention, the source of suction is incorporated within the apparatus.

Furthermore, in accordance with an embodiment of the present invention, the source of suction is external to the apparatus, the apparatus including a tube to connect the at least one port to the source of suction.

Furthermore, in accordance with an embodiment of the present invention, a thickness of the stabilizing cap is sufficient to prevent contact of a moving part of the apparatus with the surface.

Furthermore, in accordance with an embodiment of the present invention, a surface of the stabilizing cap is convex or corrugated.

Furthermore, in accordance with an embodiment of the present invention, the cutting element includes a blade.

Furthermore, in accordance with an embodiment of the present invention, the blade is configured to rotate about the stabilizing cap when making the incision.

Furthermore, in accordance with an embodiment of the present invention, the blade is curved.

Furthermore, in accordance with an embodiment of the present invention, the blade is substantially circular or semicircular.

Furthermore, in accordance with an embodiment of the present invention, the cutting element includes a stylus that is configured to rotate about the stabilizing cap when making the incision.

Furthermore, in accordance with an embodiment of the present invention, the cutting head is mounted to a handle.

Furthermore, in accordance with an embodiment of the present invention, the handle includes a user-operable control.

Furthermore, in accordance with an embodiment of the present invention, the stabilizing cap includes a protuberance.

There is further provided, in accordance with an embodiment of the present invention, a method for making an incision in a surface, the method including: placing a stabilizing cap of a cutting head of an incision apparatus on the surface so as to inhibit relative motion between the cutting head and the surface; and operating a cutting element of the cutting head, the cutting element being radially distal to the stabilizing cap, so as to make the incision such that the incision is radially distal to and at least partially surrounds the stabilizing cap.

Furthermore, in accordance with an embodiment of the present invention, the method further includes applying suction to the stabilizing cap or inserting a protuberance of the stabilizing cap into the surface when the stabilizing cap is placed on the surface.

Furthermore, in accordance with an embodiment of the present invention, placing the stabilizing cap includes insertion of the cutting head to the surface via an incision in a cornea of an eye.

### BRIEF DESCRIPTION OF THE DRAWINGS

In order for the present invention, to be better understood and for its practical applications to be appreciated, the following Figures are provided and referenced hereafter. It should be noted that the Figures are given as examples only and in no way limit the scope of the invention. Like components are denoted by like reference numerals.

FIG. 1A is a schematic illustration of an incision apparatus with a stabilizing cap, in accordance with an embodiment of the present invention.

FIG. 1B is a schematic side view of an enlargement of a cutting head of the incision apparatus shown in FIG. 1A.

FIG. 1C shows a schematic view of the incision-making side of the cutting head shown in FIG. 1B.

FIG. 2 schematically illustrates a configuration of a cutting head of an incision apparatus with a non-circular stabilizing cap, in accordance with an embodiment of the present invention.

FIG. 3 schematically illustrates a configuration of a cutting head of an incision apparatus with a non-circular stabilizing cap having multiple suction ports and a curved cutting element, in accordance with an embodiment of the present invention.

FIG. 4 schematically illustrates a configuration of a cutting head of an incision apparatus with a cutting element that surrounds the stabilizing cap, in accordance with an embodiment of the present invention.

FIG. 5 schematically illustrates a configuration of a cutting head of an incision apparatus with a heating cutting element that surrounds the stabilizing cap, in accordance with an embodiment of the present invention.

FIG. 6 schematically illustrates a variant of the cutting head shown in FIG. 1C in which the stabilizing cap does not include a suction port.

FIG. 7 schematically illustrates a variant of the cutting head shown in FIG. 1C in which the stabilizing cap includes a protuberance.

FIG. 8 schematically illustrates a variant of the cutting head shown in FIG. 2 in which the cutting element includes a stylus.

FIG. 9 schematically illustrates a variant of the cutting head shown in FIG. 1B with a stabilizing cap having a convex surface.

FIG. 10 schematically illustrates a variant of the cutting head shown in FIG. 9 in which the stabilizing cap includes a surface with corrugations.

FIG. 11 schematically illustrates an incision apparatus with an internal source of suction, in accordance with an embodiment of the present invention.

FIG. 12 schematically illustrates an incision apparatus with an external source of suction, in accordance with an embodiment of the present invention.

FIG. 13 is a flowchart depicting a method for making an incision, in accordance with an embodiment of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, us of the conjunction “or” as used herein is to be understood as inclusive (any or all of the stated options).

In accordance with an embodiment of the present invention, an apparatus for making an incision in a surface includes a cutting head that includes a stabilizing cap. A cutting element for making the incision, at least when in operation, is located radially distal to the stabilizing cap. Thus, the cutting element is configured make an incision that is radially distal to, and at least partially surrounds, the stabilizing cap. A cutting element or incision is herein referred to as being distal to the stabilizing cap when the entire cutting element (at least when in operation) or all of the incision is located outside the perimeter of the stabilizing cap. The incision is herein referred to as at least partially surrounding the stabilizing cap when the incision has a finite length and a curvature that is viewed as concave from the stabilizing cap.

The cutting element may include a blade or other cutting component (e.g., laser, heating wire, stylus, point, or other cutting component) that is configured to move in a (e.g., circular, arced, or other curved or bent) path that at least partially surrounds the stabilizing cap. Alternatively or in addition, the cutting element may include a curved or bent cutting component such that the cutting element is configured to make an incision that at least partially surrounds the stabilizing cap without being moved. The stabilizing cap is configured to inhibit (herein referring to reducing, eliminating, or preventing) relative motion between the cutting element and the surface to be cut.

When in use to make an incision in the surface, the cutting head of the apparatus is placed on the surface such that the stabilizing cap is in contact with the surface. The stabilizing cap may be configured to inhibit relative motion by application of friction forces that inhibit relative lateral motion between the stabilizing cap and the surface. For example, part or all of an inner surface of the stabilizing cap (herein referring to the surface of the stabilizing cap that is oriented toward the surface) may be made of a flexible material (e.g., a flexible rubber or plastic) that may conform of adapt to the contours of the surface. Adaptation to the contours of the surface may facilitate effective application of friction forces. For example, all or part of the stabilizing cap may be flexible, or at least a lip at the outer perimeter the stabilizing cap may be flexible. The shape of the stabilizing cap may be adapted to that of a surface on which the stabilizing cap is configured to be placed. For example, if the surface to be cut is convex, the inner surface of the stabilizing cap may be made concave so as to approximately match the contours of the surface. In some cases, the inner surface of the stabilizing cap may be flat, convex, or otherwise shaped, formed, or textured (e.g., with corrugations, bosses, or otherwise).

In some cases, friction between the stabilizing cap and the surface may be enhance by application of suction within a space that is formed between the inner surface of the stabilizing cap and the surface to be cut. For example, the inner surface of the stabilizing cap may include one or more openings or ports that connect the space to a source of suction. The source of suction may be self-contained within the incision apparatus (e.g., within a handle), or may be external to the apparatus (e.g., and connected to the suction ports by a tube or conduit). Alternatively or in addition, flexibility of the stabilizing cap may enable the stabilizing cap to act as a suction cup. In this case, slight pressing of the stabilizing cap against the surface may remove air from the space between the inner surface and the surface to be cut.

The stabilizing cap may provide spacing to prevent contact between a moving part of the incision apparatus and the surface being cut. For example, a cutting element (e.g., blade, stylus, or other cutting element) may be mounted on a moving (e.g., rotating) arm disk or other moving structure. The stabilizing cap may have sufficient thickness (herein referring to a dimension that is substantially perpendicular to the surface to be cut when the incision apparatus is placed for making an incision in the surface) to ensure that only the cutting element itself (e.g., blade edge, point, heated wire, or other cutting component) contacts that surface. Thus, the stabilizing cap may prevent possible tearing or dragging of the surface (e.g., a flexible surface such as the lens capsule in the eye) by a moving part (e.g., an arm or disk that rotates the cutting element) other than the cutting element.

Alternatively or in addition, the inner surface of the stabilizing cap may include one or more protuberances (e.g., in the form of texturing, spikes, cleats, fins, pins, projections, or similar structure) whose ends may contact or insert in to the surface to be cut. The contact of the structure with the surface may contribute to inhibition of the relative motion. The stabilizing cap may include a combination of the above.

The stabilizing cap and cutting element are incorporated into a cutting head of the apparatus. The cutting head may be made sufficiently small to enable insertion through an opening to the surface to be cut. For example, when the surface to be cut is the anterior surface of the eye lens, the cutting head may be sufficiently small (e.g., its longest dimension no longer than a few, e.g., under five, millimeters) to enable insertion through a minimal opening in the cornea of the eye.

The cutting head is typically mounted on a handle. For example, the handle may be shaped so as to enable accurate manipulation of the cutting head while holding the handle in a convenient and comfortable manner. For example, the cutting head may extend from the handle in a direction described by an angle (e.g., approximately equal to 40°), having a general form that is similar in shape to that of an ice hockey stick.

The end of the handle near the cutting head (herein referred to as the distal end, as being distal to place on the handle where the handle is held when the apparatus is in use) may have a sufficiently small cross section to enable insertion through an opening together with the cutting head.

A proximal section end of the handle may be wider than the distal end. For example, the proximal section may be sufficiently wide to enable secure and controllable grasping and manipulation of the handle, and thus of the cutting head. The proximal section of the handle may be sufficiently wide so as to house various components for operation of the apparatus.

For example, the handle may house a mechanical component that may be manipulated (e.g., a lever, slide, knob, or other mechanical component) to operate a rotating or otherwise moving cutting element. The handle may house an electric motor and battery to operate the cutting element. The handle may house a transmission that transmits mechanical motion on the proximal section of the handle to the cutting head. For example, the transmission may include a string, wire, or cord that extends from the mechanical component or the motor to the cutting head. When the end of the string in the handle is pulled by the mechanical component or the motor, the end that is connected to the cutting head moves the cutting element. In the case of a rotating cutting element, the end of the string in the cutting head may wrap around a pulley to which the cutting element is attached. The handle may house a power supply and related circuitry or electronics for operation of a heating element in the cutting head (and wiring for conducting electric current from the power supply to the heating element). The handle may house a mechanically or electrically operated pump for providing suction to the stabilizing cap (and tubing for conducting the suction from the pump to the stabilizing cap). The handle may include a connector for connecting to an external power supply or to an external suction pump.

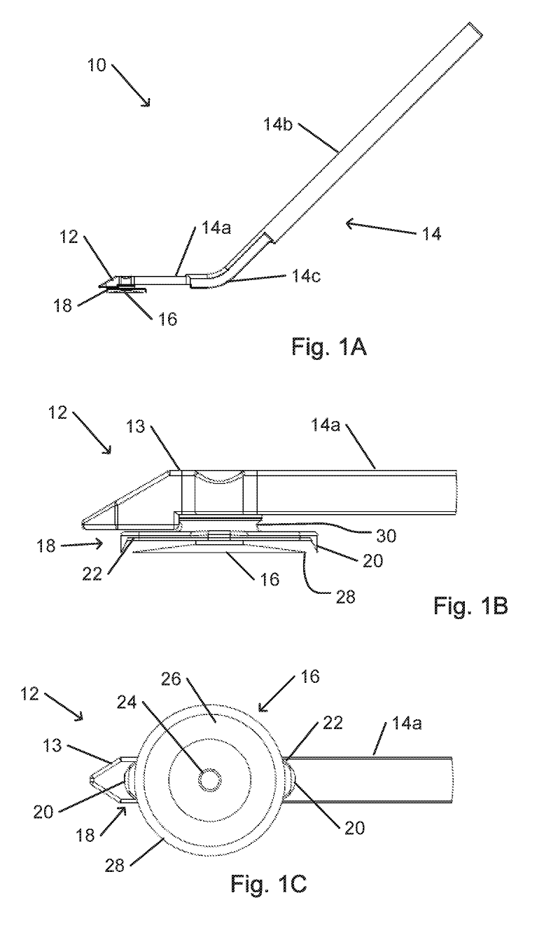

FIG. 1A is a schematic illustration of an incision apparatus with a stabilizing cap, in accordance with an embodiment of the present invention. FIG. 1B is a schematic side view of an enlargement of a cutting head of the incision apparatus shown in FIG. 1A. FIG. 1C shows a schematic view of the incision-making side of the cutting head shown in FIG. 1B.

Incision apparatus 10 includes a cutting head 12 attached to distal end 14a of apparatus handle 14. Proximal section 14b of apparatus handle 14 is configured for holding by an operator of incision apparatus 10. Apparatus handle 14 may include a bend 14c. Bend 14c may enable convenient holding of proximal section 14b when distal end 14a and cutting head 12 are placed on a surface to be cut.

Cutting head 12 includes stabilizing cap 16 and incision assembly 18.

Stabilizing cap 16 includes circular lip 28 surrounding inner surface 26. Inner surface 26 may be concave, or may be adaptable to contuours of a surface to be cut. Cap lip 28 may be placed against a surface in which an incision is to be made. In the example shown in FIG. 1C, cap lip 28 is substantially circular. Inner surface 26 of stabilizing cap 16 is configured to conform to a flat or convex surface against which stabilizing cap 16 is placed. For example, the surface may include a convex anterior surface of a lens of an eye.

In some cases, stabilizing cap 16 may include a suction port 24. Suction port 24 may open to one or more tubes, conduits, or channels that connect to a suction source. The tubes may be internal to apparatus handle 14. The suction source may include, for example, a manually operated or electrically powered suction pump. The suction source may be internal to, or external to, apparatus handle 14 or another part of incision apparatus 10.

Incision assembly 18 includes one or more cutting elements 20. For example, incision assembly 18 may include two cutting elements. For example, a cutting element 20 may include a blade (e.g., a convex blade) that may cut a surface when cutting element 20 is drawn across that surface.

Cutting elements 20 may be mounted on cutting arm 22. For example, two cutting elements 20 may be mounted at or near opposite ends of a cutting arm 22 in the form of a linear bar. Cutting arm 22 may be moved to draw cutting elements 20 across the surface to be cut.

In the example shown, cutting arm 22 may be rotated around arm axis 30, or together with the arm axis 30, to make a circular incision that surrounds stabilizing cap 16. For example, arm axis 30, cutting head housing 13, or both may house components of a cutting mechanism to rotate cutting arm 22. The cutting mechanism may also extend cutting arm 22 and attached cutting elements 20 outward toward the surface to be cut.

In some cases, stabilizing cap 16 (e.g., with applied suction) may assist in removing a piece of the surface (e.g., a circular section) that was severed from the remainder of the surface by operation of incision assembly 18.

Various alternatives to the configuration of cutting head 12 shown in FIGS. 1B and 1C are possible.

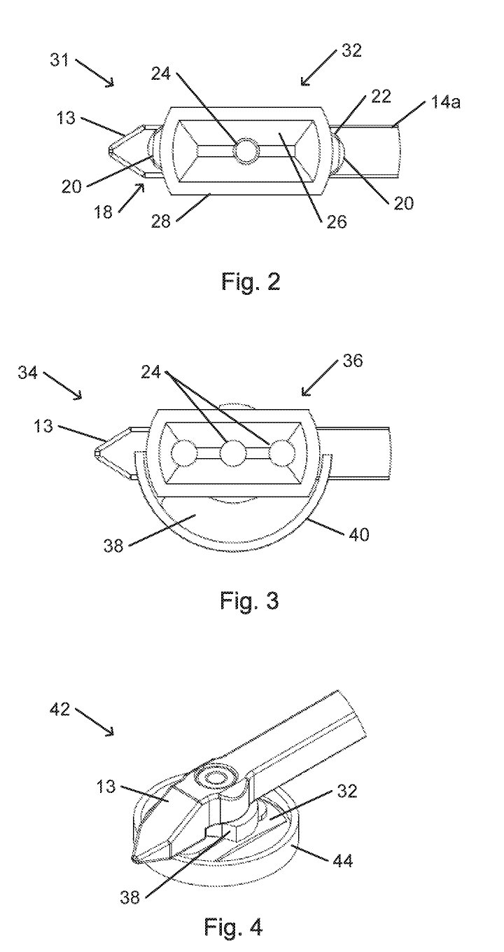

FIG. 2 schematically illustrates a configuration of a cutting head of an incision apparatus with a non-circular stabilizing cap, in accordance with an embodiment of the present invention.

Cutting head 31 includes noncircular stabilizing cap 32. Incision assembly 18 is configured to rotate cutting elements 20 about noncircular stabilizing cap 32.

For example, noncircular stabilizing cap 32 may have the approximate shape of a section of a circle that is bounded by two parallel chords that are equidistant from the center of the circle. As other examples, a noncircular stabilizing cap may have a form of a polygon or another shape with curved or straight sides.

FIG. 3 schematically illustrates a configuration of a cutting head of an incision apparatus with a non-circular stabilizing cap having multiple suction ports and a curved cutting element, in accordance with an embodiment of the present invention.

Cutting head 34 includes noncircular stabilizing cap 36. Noncircular stabilizing cap 36 includes a plurality of suction ports 24. The example shown includes three suction ports 24. However, noncircular stabilizing cap 36 may include fewer or more than three suction ports 24. For example, suction ports 24 may all be connected to a single suction source, or some of suction ports 24 may be connected to two or more different suction sources.

Cutting head 34 includes curved cutting element 40. For example, curved cutting element 40 may include a blade that is bent in the form of a circular arc, or another curved shape. In the example shown, curved cutting element 40 includes an approximately semicircular blade. In some cases, a curved cutting element may include two or more spatially separated curved blades (e.g., all of the blades being bent in the form of arcs of a single circle, with at least one of the blades being bent in the form of a circular arc that is smaller than a semicircle). In some cases, a curved cutting element may include curved blades in the form of arcs of concentric circles of different diameters (e.g., in order to produce a set of concentric incisions). In some cases, a curved cutting element may include a blade that is bent in the form of a curve other than a circular arc.

Cutting mechanism 38 may be configured to rotate curved cutting element 40 around noncircular stabilizing cap 36, to extend curved cutting element 40 toward or into a surface in which and incision is to made, or both.

Other combinations of features are possible. For example, curved cutting element 40 may be included in a cutting head having a circular stabilizing cap, or having a noncircular stabilizing cap with a single suction port 24.

FIG. 4 schematically illustrates a configuration of a cutting head of an incision apparatus with a cutting element that surrounds the stabilizing cap, in accordance with an embodiment of the present invention.

Cutting head 42 includes noncircular stabilizing cap 32. Cutting head 42 includes surrounding cutting element 44. For example, surrounding cutting element 44 may include a blade that is bent in the form of a circle that surrounds noncircular stabilizing cap 32. In the example shown, surrounding cutting element 44 includes an approximately circular blade. In some cases, a surrounding cutting element may include a noncircular blade (e.g., polygonal or oval).

Cutting mechanism 38 may be configured to rotate surrounding cutting element 44 around noncircular stabilizing cap 32, to extend surrounding cutting element 44 toward or into a surface in which and incision is to made, or both.

Other combinations of features are possible. For example, surrounding cutting element 44 may be included in a cutting head having a circular stabilizing cap.

In some cases, the cutting element may include a component that makes an incision without use of a blade.

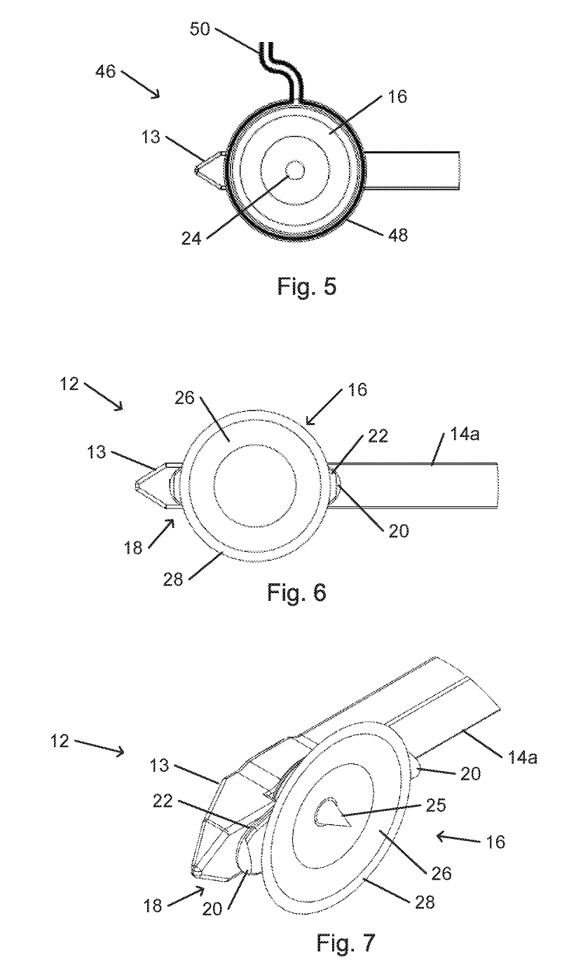

FIG. 5 schematically illustrates a configuration of a cutting head of an incision apparatus with a bladeless cutting element that surrounds the stabilizing cap, in accordance with an embodiment of the present invention.

Cutting head 46 includes stabilizing cap 16. Cutting head 46 includes bladeless cutting element 48. For example, bladeless cutting element 48 may include a heatable component (e.g., an electrically heatable wire). As another example, bladeless cutting element 48 may operate by cooling, by fluid jets, by suction, by light (e.g., laser light), chemically, or otherwise.

Bladeless cutting element 48 at least partially surrounds stabilizing cap 16. In the example shown, bladeless cutting element 48 includes an approximately circular cutting component. In some cases, a bladeless cutting element 48 may include a heatable or otherwise operable component that may be rotated about stabilizing cap 16.

Bladeless cutting element 48 may include a supply line 50 that provides electrical power or a material for operation of bladeless cutting element 48. For example, supply line 50 may include an electrical cable that may be connected to an electrical power source (e.g., within or external to the incision apparatus) to provide electrical power to heat a heatable component or other electrical component of bladeless cutting element 48. Supply line 50 may supply a coolant, a fluid, a chemically active material, a connection to a suction source, or other material flow used in operation of bladeless cutting element 48.

For example, when a heated bladeless cutting element 48 is placed against a suitable surface, the heated component may form a cut or incision in the surface.

In some cases, a stabilizing cap may not include a suction port. In some cases, the stabilizing cap may include one or more protuberances.

FIG. 6 schematically illustrates a variant of the cutting head shown in FIG. 1C in which the stabilizing cap does not include a suction port.

Stabilizing cap 16 may be configured to inhibit relative motion between cutting head 12 and a surface to be cut by conforming to a contour of the surface, by functioning as a suction cup, or otherwise.

FIG. 7 schematically illustrates a variant of the cutting head shown in FIG. 1C in which the stabilizing cap includes a protuberance.

Protuberance 25 extends from inner surface 26 of stabilizing cap 16. For example, protuberance 25 may terminate with a point. The point may be inserted into the surface so as to prevent relative motion between stabilizing cap 16 and the surface. Such insertion may be used, for example, the incision is made to remove a section of the surface and that section is to be discarded. For example, a removed section of a lens capsule is typically discarded. In some cases, protuberance 25 may be made of a flexible material that may bend, distort, or otherwise change shape when contacting the surface. The change in shape may increase friction between stabilizing cap 16 and the surface.

A stabilizing cap may include two or more protuberances. For example, the protuberances may be distributed in a pattern on inner surface 26.

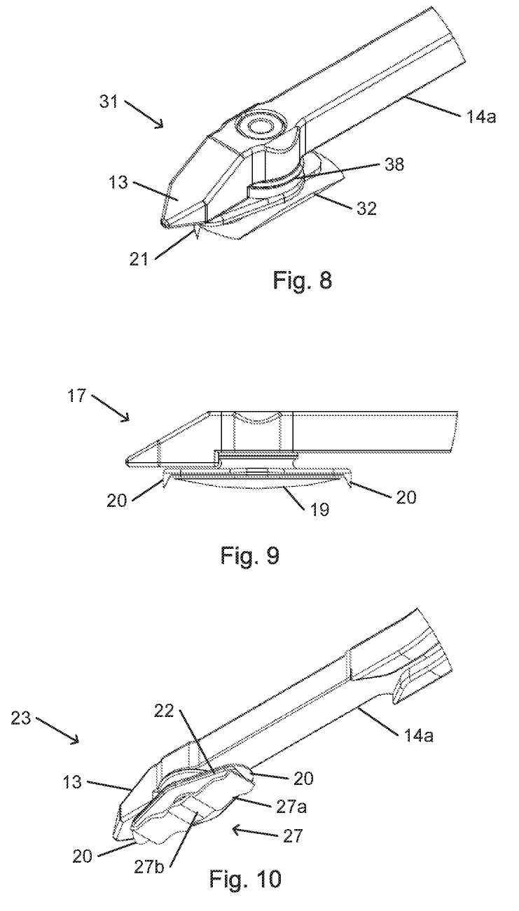

FIG. 8 schematically illustrates a variant of the cutting head shown in FIG. 2 in which the cutting element includes a stylus.

Cutting head 31 includes a cutting element in the form of stylus 21. Stylus 21 may be rotated by cutting mechanism 38 about noncircular stabilizing cap 32 to make an incision in the form of a circle or circular arc that at least partially surrounds noncircular stabilizing cap 32.

A stabilizing cap of an incision apparatus, in accordance with an embodiment of the present invention, may bew non-concave. In such a case, the stabilizing cap may be made of a flexible material that is configured to conform to contours of a surface against which the stabilizing cap is placed. Such conforming may enable sufficient friction between the stabilizing cap and the surface so as to inhibit relative movement between the stabilizing cap and the surface. In some cases, the stabilizing cap may be structured so as facilitate contact between the stabilizing cap and the surface. For example, a surface of the stabilizing cap that contacts the surface to be cut may be convex or corrugated.

FIG. 9 schematically illustrates a variant of the cutting head shown in FIG. 1B with a stabilizing cap having a convex surface.

Cutting head 17 includes convex stabilizing cap 19. Convex stabilizing cap 19 may be configured (made of flexible elastic or plastic material) to conform to (e.g., change shape to substantially complement or match) contours of a surface against which stabilizing cap 19 is placed.

FIG. 10 schematically illustrates a variant of the cutting head shown in FIG. 9 in which the stabilizing cap includes a surface with corrugations.

Cutting head 23 includes a corrugated stabilizing cap 27. Corrugated stabilizing cap 27 may included corrugations having a cross section in the form of continuous smooth waves (as in cap section 27a), or may include ridged corrugations or discontinuous waves (as in cap section 27b). The form of corrugated stabilizing cap 27 may facilitate inhibition of relative motion between corrugated stabilizing cap 27 and a surface to be cut upon which corrugated stabilizing cap 27 is placed.

Other combinations of features are possible. For example, bladeless cutting element 48 may be included in a cutting head having a noncircular, non-concave, or other type of stabilizing cap. The stabilizing cap may not include a suction port, may include more than one suction port, protuberances, corrugations, or other structure.

A suction port of a stabilizing cap of a cutting head of an incision apparatus, in accordance with an embodiment of the present invention, may connect to a source of suction that is internal to, or external to, the incision apparatus.

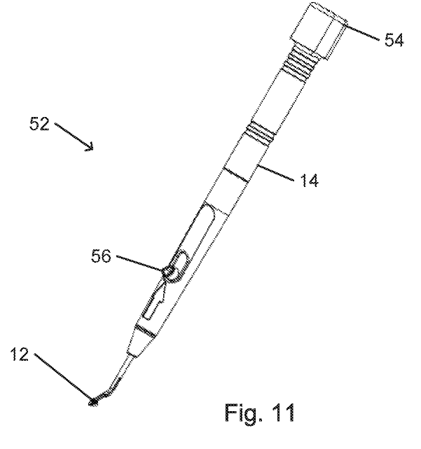

FIG. 11 schematically illustrates an incision apparatus with self-contained source of suction, in accordance with an embodiment of the present invention.

Incision apparatus 52 includes self-contained suction source 54. For example, self-contained suction source 54 may apply suction to a stabilizing cap of cutting head 12 via one or more tubes or conduits. For example, the tubes or conduits may be enclosed within apparatus handle 14.

For example, self-contained suction source 54 may include a motorized pump that generates suction. Alternatively or in addition, self-contained suction source 54 may include a manually operated pump (e.g., operable by pressing on a surface of self-contained suction source 54).

Self-contained suction source 54 may be operated at the initiative of a user of incision apparatus 52 or automatically. For example, a user-operable control 56 may be operated by the user to initiate, end, or control operation of cutting head 12. User-operable control 56 may include a single control device (e.g., mechanically, electromagnetically, optically, thermally, or otherwise operated lever, button, switch, or other control device), or may be representative of two or more such control devices.

For example, user-operable control 56 may be operable to operate a cutting mechanism of cutting head 12 (e.g., rotate or extend a cutting element). In some cases, user-operable control 56 may be separately operable (e.g., separate control device, or separate operation state) to operate self-contained suction source 54. In some cases, operation of user-operable control 56 to operate the cutting mechanism may also operate self-contained suction source 54 (e.g., prior to, or concurrently with, operation of the cutting mechanism). In some cases, operation of self-contained suction source 54 may be automatic or semi-automatic. For example, self-contained suction source 54 may operate continuously (e.g., whenever incision apparatus 52 is powered on), or in response to a sensed or detected event (e.g., contact or proximity of the stabilizing cap with or to a surface to be cut.

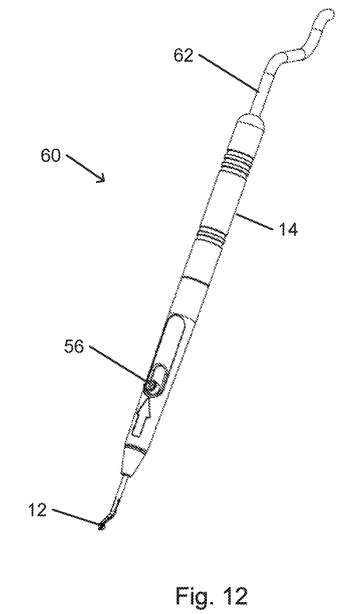

FIG. 12 schematically illustrates an incision apparatus with an external source of suction, in accordance with an embodiment of the present invention.

Incision apparatus 60 includes suction tube 62. Suction tube 62 may be connected to an external suction source. For example, an external suction source may apply suction to a stabilizing cap of cutting head 12 via suction tube 62. Suction tube 62 may include one or more separate hoses, tubes, or conduits. Suction tube 62 may enable the suction to be applied to the stabilizing cap via one or more tubes, hoses, or conduits, e.g., that are enclosed within apparatus handle 14.

For example, an external suction source may include a motorized or mechanically (e.g., hand- or foot-operated) operated pump that may be operated to generate suction.

The external suction source may be operated at the initiative of a user of incision apparatus 60, may operate continuously, or may operate automatically. For example, a user-operable control 56 may be operated by the user to initiate, end, or control operation of the external suction source. Alternatively or in addition, user-operable control 56 may be operated by the user to control in internal valve or regulator (e.g., encased within apparatus handle 14) that may be operated to initiate, terminate, or control suction that is applied to a stabilizing cap of cutting head 12.

In some cases, user-operable control 56 may be operable to control application of suction from the external suction source to the stabilizing cap. In some cases, operation of user-operable control 56 to operate the cutting mechanism may also control application of the suction to the stabilizing cap (e.g., prior to, or concurrently with, operation of the cutting mechanism). In some cases, application of the suction to the stabilizing cap may be automatic or semi-automatic. For example, the suction may be applied continuously (e.g., whenever incision apparatus 60 is powered on), or in response to a sensed or detected event (e.g., contact or proximity of the stabilizing cap with or to a surface to be cut.

An incision apparatus, in accordance with an embodiment of the present invention, may be utilized in a method to make an incision.

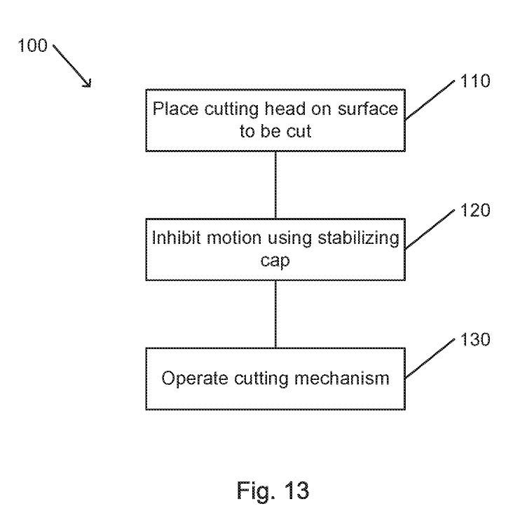

FIG. 13 is a flowchart depicting a method for making an incision, in accordance with an embodiment of the present invention.

Incision method 100 may be performed by a user who uses an incision apparatus to make an incision.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

A cutting head of the incision apparatus may be placed on a surface to be cut (block 110). For example, the cutting head may be inserted through an incision in the cornea of an eye and placed on the anterior surface of a lens of the eye, or may be placed on another surface to be cut.

Relative motion between the cutting head and the surface may be inhibited by use of the stabilizing cap of the cutting head (block 120). For example, operation of the stabilizing cap may prevent relative motion between an eye lens and the cutting head. In some cases, placement of the stabilizing cap on the surface may prevent or inhibit relative motion via friction forces. The stabilizing cap may be configured to conform or engage the surface so as to facilitate application of friction forces. In some cases, a protuberance from the stabilizing cap may pierce or insert into the surface so as to engage the surface. In some cases, suction may be applied to the stabilizing cap. For example, light pressing of the cutting head with a flexible stabilizing cap against the surface cause the stabilizing cap to act as suction cup to generate suction. As another example, a self-contained or external suction source may be operated to apply suction to the stabilizing cap.

A cutting mechanism of the incision apparatus may be operated to make the incision (block 130). For example, one or more blades or other cutting elements may rotate about the stabilizing cut to make the incision. One or more cutting elements that fully or partially surround the stabilizing cap may be pressed into the surface. One or more heating cutting elements may be heated to make the incision.

During the operation of the cutting mechanism, the stabilizing cap may prevent contact between a moving part of the cutting mechanism (other than the cutting element itself) and the surface.

After the incision is made, part or all of the surface, or of connected or nearby structure, may be removed. For example, the interior of an eye lens may be removed via the incision.

Different embodiments are disclosed herein. Features of certain embodiments may be combined with features of other embodiments; thus certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An apparatus for making an incision in a surface, the apparatus comprising a cutting head that includes:
a stabilizing cap for placement on the surface and operable to inhibit relative motion between the cutting head and the surface; and
a cutting element that is located radially distal to the stabilizing cap and that is configured to make the incision which is radially distal to and at least partially surrounds the stabilizing cap.

2. The apparatus of claim 1, wherein a surface of the stabilizing cap for placement on the surface is concave.

3. The apparatus of claim 1, wherein the stabilizing cap is substantially circular.

4. The apparatus of claim 1, wherein the stabilizing cap has a noncircular shape.

5. The apparatus of claim 1, wherein the stabilizing cap comprises at least one port that is connectable to a source of suction.

6. The apparatus of claim 5, wherein the source of suction is incorporated within the apparatus.

7. The apparatus of claim 5, wherein the source of suction is external to the apparatus, the apparatus including a tube to connect said at least one port to the source of suction.

8. The apparatus of claim 1, wherein a thickness of the stabilizing cap is sufficient to prevent contact of a moving part of the apparatus with the surface.

9. The apparatus of claim 1, wherein a surface of the stabilizing cap is convex or corrugated.

10. The apparatus of claim 1, wherein the cutting element comprises a blade.

11. The apparatus of claim 10, wherein the blade is configured to rotate about the stabilizing cap when making the incision.

12. The apparatus of claim 10, wherein the blade is curved.

13. The apparatus of claim 12, wherein the blade is substantially circular or semicircular.

14. The apparatus of claim 1, wherein the cutting element comprises a stylus that is configured to rotate about the stabilizing cap when making the incision.

15. The apparatus of claim 1, wherein the cutting head is mounted to a handle.

16. The apparatus of claim 15, wherein the handle includes a user-operable control.

17. The apparatus of claim 1, wherein the stabilizing cap comprises a protuberance.

18. A method for making an incision in a surface, the method comprising:
placing a stabilizing cap of a cutting head of an incision apparatus on the surface so as to inhibit relative motion between the cutting head and the surface; and
operating a cutting element of the cutting head, the cutting element being radially distal to the stabilizing cap, so as to make the incision such that the incision is radially distal to and at least partially surrounds the stabilizing cap.

19. The method of claim 18, further comprising applying suction to the stabilizing cap or inserting a protuberance of the stabilizing cap into the surface when the stabilizing cap is placed on the surface.

20. The method of claim 18, wherein placing the stabilizing cap comprises insertion of the cutting head to the surface via an incision in a cornea of an eye.
